(19) United States
(12) Patent Application Publication
(10) Pub. No.: US 2007/0038171 A1
(43) Pub. Date: Feb. 15, 2007

(54) Shunt system

(76) Inventor: Peter L. Mayer, Sarasota, FL (US)

Correspondence Address: PAUL D. BIANCO: FLEIT, KAIN, GIBBONS,;GUTMAN, BONGINI, & BIANCO P.L., 21355 EAST DIXIE HIGHWAY, SUITE 115, MIAMI, FL, 33180, US

(21) Appl. No.: 11/188,675

(22) Filed: Jul. 25, 2005

(51) Int. Cl. A61M 5/00

(52) U.S. Cl. 604/9

(57) ABSTRACT

The present application provides a subcutaneously implantable device for removing fluid from a first location in a body of a patient. The device includes a fluid controller having a fluid inlet and a fluid outlet. The fluid controller is continually operable between a first and second cycle, wherein in the first cycle the fluid controller draws fluid in through the fluid inlet and in the second cycle the fluid controller expels a first portion of the fluid out through the fluid inlet and a second portion of the fluid out through the fluid outlet.

### FIELD OF THE INVENTION

The invention relates to a medical device and method therefore, and more particularly to a shunt system for controlling cerebrospinal fluid pressure in a cranial space.

### BACKGROUND OF THE INVENTION

In the medical arts, to relieve undesirable accumulation of fluids it is frequently necessary to drain the fluids from one part of the human body to another in a controlled manner. This can be required, for example, in the treatment of hydrocephalus, which is caused by excess cerebrospinal fluid accumulating inside the head. Congenital hydrocephalus may result in excessive skull enlargement and, if untreated, progress to brain damage, or even death. When the condition occurs later in a person's life, the skull is no longer flexible and the condition can cause headaches, vomiting, and loss of coordination and mental functioning.

In treating hydrocephalus, cerebrospinal fluid is drained from the cranial space utilizing a drainage or shunt system. A shunt system typically includes a catheter inserted into the ventricle through the skull. The catheter is connected to a tube though which excess cerebrospinal fluid may be removed from the brain and reintroduced into another portion of the body of the patient, such as the peritoneal cavity or the vascular system.

To control the flow of cerebrospinal fluid and maintain the proper pressure in the brain ventricle, a valve can be positioned on the tube. The valves are generally one way, only allowing fluid to pass out of the cranial space. The valve is designed to open due to slight differential pressure between the inlet or proximal end of the shunt and its outlet or distal end. The valve will close in the event the pressure differential reverses, which may occur by coughing or straining of the patient, thereby preventing a reverse flow fluid through the shunt into the ventricular cavity. Alternatively, a pump can be positioned on the tube, which can operate to draw fluid from the cranial space.

### SUMMARY OF THE INVENTION

The present application provides a subcutaneously implantable device for removing fluid from a first location in a body of a patient. The device includes a fluid controller having a fluid inlet and a fluid outlet. The fluid controller is operable between a first and second cycle, wherein in the first cycle the fluid controller draws fluid in through the fluid inlet and in the second cycle the fluid controller expels a first portion of the fluid out through the fluid inlet and a second portion of the fluid out through the fluid outlet.

The device further includes a first catheter having a first end positionable within the first location in the body of the patient and a second end connected to the fluid inlet. A second catheter includes a first end connected to the fluid outlet and a second end through which the fluid is expelled. A one way valve interposed, for example, between the fluid outlet and the second catheter prevents fluid from entering, and possibly also exiting, the fluid controller though the fluid outlet during the first cycle.

The fluid controller can include a reservoir in fluid communication with the first and second catheters and a pump connected thereto. The pump is operable between the first and second cycles. In the first cycle excess fluid is drawn in through the first catheter into the reservoir and in the second cycle some of the fluid is forced out of the reservoir through the first catheter back into the first location, while some is forced out of the reservoir through the one-way valve and second catheter so that the net effect is a reduction in accumulated fluid or pressure. The return of some of the fluid through the first catheter may help clear debris or particulate matter that may collect inside the device or its components (e.g., filters, inlets, the reservoir, etc.).

In a method of draining a fluid from the first location in the body of the patient the device is positioned in fluid communication with the first location in the body of the patient. Next, fluid is drained from the first location in the body of the patient into the device. The fluid is then expelled from the device, wherein the first portion of the fluid is expelled from the device back into the first location in the body of the patient and a second portion of the fluid is expelled from the device to a second location different from the first location in the body of the patient. The device may be operated continuously or periodically between the first and second cycles until the appropriate amount of fluid is removed, until a desired pressure is achieved or maintained in the treated area of the patient, or when other patient conditions are met.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 depicts a shunt system of the present invention;

FIG. 2 is a schematic representation of a fluid controller of the shunt system of FIG. 1;

FIGS. 3A-B are schematic representations of a use of the shunt system of FIG. 1;

FIG. 4 is a schematic representation of another fluid controller of the shunt system of FIG. 1;

FIG. 5 depicts an exemplary pump of the shunt system of FIG. 1;

FIGS. 6A-B are schematic representations of a use of the exemplary pump of FIG. 5.

### DETAILED DESCRIPTION OF THE INVENTION

The present invention is directed to a shunt system which is responsive to the accumulation of fluid or the build up of pressure in the cranial space. The shunt system includes a fluid controller for removal of the excessive fluid in a controlled manner.

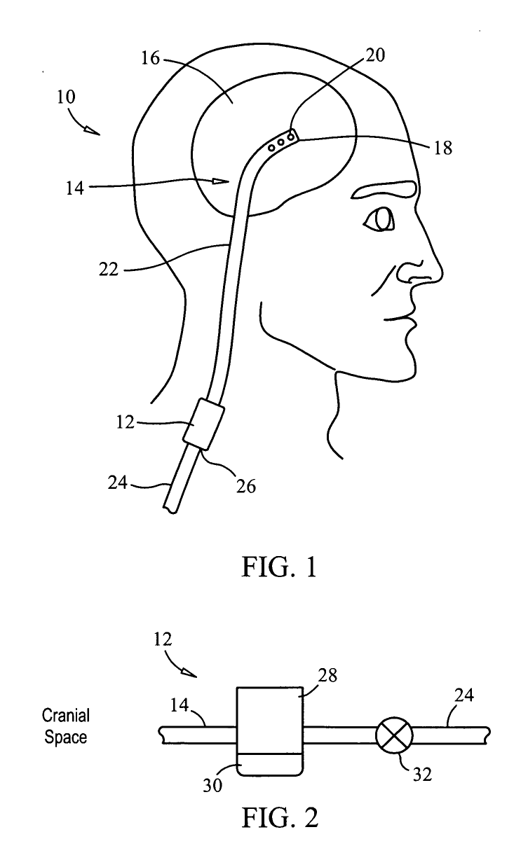

Referring now to the drawing figures in which like reference designators refer to like elements, there is shown in FIG. 1 a shunt system 10 having a fluid controller 12, a first catheter 14, and a second catheter 24. The first catheter can be positioned so that a first end 18 is disposed in a cranial space 16 of a patient. The first end 18 has one or more openings or ports 20 through which the cerebrospinal fluid can be drawn into the first catheter 14. For instance, the first end 18 may terminate with a single opening or port 20, or alternatively may include a plurality of ports 20. A second end 22 of the first catheter 14 is in fluid communication with the fluid controller 12.

The shunt system 10 further includes a second catheter 24 having a first end 26 in fluid communication with the fluid controller 12. The second end (not shown) of the second catheter 24 can be positioned in a portion of the body of the patient which can accept and/or expel the excess fluid.

Referring to FIG. 2, the fluid controller 12 includes a fluid reservoir 28 in fluid communication with the first and second catheters 14, 24. A pump 30 is operably connected to the fluid reservoir 28 and is configured to both draw a vacuum in and pressurize the fluid reservoir 30. A one-way valve 32 is interposed between the fluid reservoir 28 and the second catheter 24. The one-way valve 32 is configured to allow fluid to exit the fluid reservoir 28 through the second catheter 24, yet prevent fluid from being drawn into the fluid reservoir 28 through the second catheter 24.

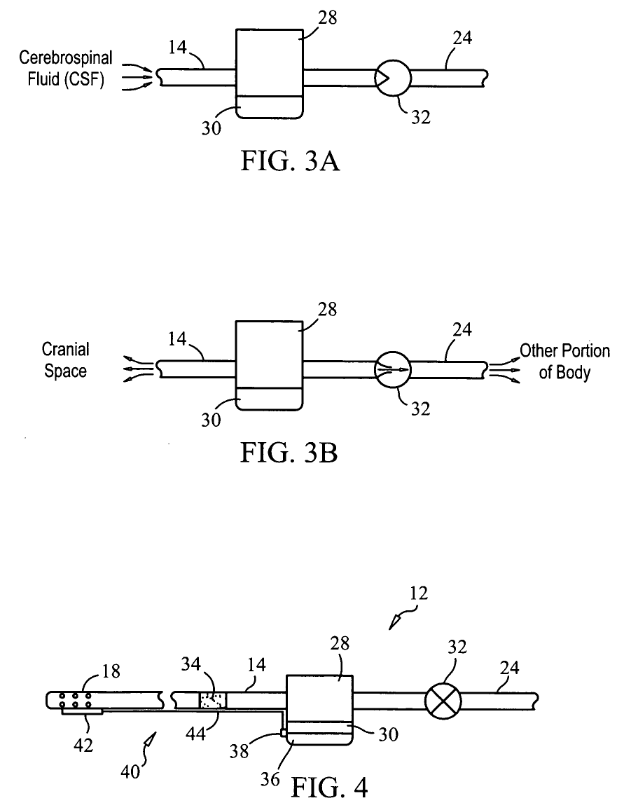

FIGS. 3A and 3B illustrate the use of the device during steps or cycles of drawing fluid into the device and expelling fluid from it. Referring to FIG. 3A, the pump 30 is activated to draw a vacuum in the fluid reservoir 28, such that the pressure in the fluid reservoir 28 is less than the pressure in the cranial space 16. As a result, fluid is drawn into the fluid reservoir 28 from the cranial space 16 through the first catheter 14. In a likewise manner, the pressure in the fluid reservoir 28 may be less than the pressure in the second catheter 24, and if so, the one-way valve 32 will be maintained in a closed position in order to prevent fluid from entering or exiting the reservoir 28 through the second catheter 24.

Referring to FIG. 3B, in a second cycle the pump 30 pressurizes the fluid reservoir 28, such that the pressure in the fluid reservoir 28 is greater than the pressure in the cranial space 16 and the second catheter 24. The pressure in the fluid reservoir 28 is sufficiently high to open the one-way valve 32. In this manner, the fluid is forced out of the fluid reservoir 28, where a portion of the fluid is forced out through the second valve 32 and the second catheter 24.

Additionally, some fluid also may be forced out of the fluid reservoir 28 through the first catheter 14, back into the cranial space 16. In this manner, the first catheter 14 or other upstream components of the device may be back flushed, which can substantially prevent the accumulation of particulate matter in the first catheter 14, reservoir 28, or other parts of the device. When activated, the pump 28 operates between the first and second cycles to remove fluid from the cranial space 16.

Operation of the pump may be configured so that it runs continuously, only when certain conditions are present, or only until certain conditions are met. For example, the reservoir may be maintained at or below a certain pressure during the first cycle or step, or within a pressure range, so that any build up of pressure in the cranial space causes fluid to be drained into the reservoir. The frequency and/or duration of this first cycle may be a predetermined time or may vary based upon meeting one or more desired conditions. For instance, low pressure may be used to draw in fluid on a regular, repeating frequency, or may last a predetermined length of time.

Alternatively, however, the reservoir may accumulate the excess fluid over time until reaching a triggering event, such as the reservoir becoming full, exceeding a certain pressure threshold, or both. Upon reaching the end of a predetermined time or upon satisfying a triggering event, the reservoir then undergoes the second cycle or step of evacuating fluid from the reservoir. As above, this second cycle may last a predetermined length of time, may occur on a regular, periodic frequency, or may continue until reaching a triggering event.

For example, the second cycle may continue until the reservoir is substantially free of fluid, or at least until it is about half full or less. Alternatively, the second cycle may continue until the pressure in the reservoir or in the treated area of the patient reaches or falls below a certain level so that, upon concluding the second cycle (and possibly reverting to a first cycle) the pressure in the reservoir or patient is within a desired pressure range or below a threshold pressure level.

In another embodiment, the fluid controller may be configured to continuously alternate between first and second cycles with regular frequency and duration, regardless of the amount of accumulated fluid in the reservoir. The duration and frequency of the cycles may be selected or adjusted by the physician to correspond to the patient's medical condition and rate of fluid accumulation.

Additionally, the fluid controller may be configured to periodically operate between the first and second cycles. For instance, the fluid controller may only cause the device to operate in the first or second cycles once a certain pressure level is reached, or after a period of inactivity.

Referring to FIG. 4, a filter 34 can be positioned in the first catheter 14, or in the alternative, in an inlet of the fluid reservoir 28. The filter 34 is dimensioned to prevent or reduce the likelihood of particulate matter entering the fluid reservoir 28. Additionally, when the pump 30 is operated in the second cycle, the portion of the fluid that is forced back through the first catheter 14 substantially removes the particulate matter from the filter 34, back into the cranial space 16. This continual back flushing of the filter 34 and first catheter 14 substantially prevents the accumulation of particulate matter in the filter 34 or in the first catheter 14 that might obstruct or block fluid flow to the device.

The fluid controller 12 can further include a power supply 36 operably connected to the pump 30. The power supply 36 may also include a switch 38 which can be actuated between an “ON” position, in which power is supplied to the pump 30, and an “OFF” position, in which power is not supplied to the pump 30.

The power supply 36 can be a self-contained power source, such as a high-capacity battery such as already widely used in pacemakers, stimulators, defibrillators and the like. The battery 36 can be located external to fluid controller 12 and inserted in subcutaneous tissue to provide easy access for replacement in the event of failure. It is also contemplated, however, that battery 36 could be integrally housed with fluid controller 12.

In an exemplary embodiment, the switch 38 can be a manual switch which can be operated by the patient or medical practitioners. Manual switches can include magnetic switches, toggle switches, depression switches, RF switch, etc. For example, for a magnetic switch a first magnetic field can be placed in proximity to the magnetic switch. The first magnetic field actuates the magnetic switch from a first position to a second position, switching the magnetic switch into the “ON” position. Similarly, a second magnetic field can be placed in proximity to the magnetic switch. The second magnetic field actuates the magnetic switch from the second position back to the first position, switching the magnetic switch into the “OFF” position. The first and second magnetic fields can have different polarities, or, in the alternative the same polarity.

In addition to a switch having an “ON” and “OFF” position, it also may have a third position or state where the fluid control system determines when to operate the device in the first or second cycle and when to cease operating in these cycles until some other condition is met (e.g., fluid pressure, fluid levels, etc.). In this alternative embodiment, the “ON” and “OFF” positions of the switch may operate as manual overrides of the fluid control system. Thus, a patient or physician may chose to have the device operate relatively independently based on triggering events that may be monitored by the device, but also may utilize the switch to ensure that the device is either off or on.

Alternatively, a toggle or depression switch can be positioned proximal to the surface of a portion of the body of the patient. The switch is positioned such that the patient or medical practitioner can physically access the switch, to actuate the switch between first and a second “ON” and “OFF” positions. In this manner the patient or medical practitioner can control the operation of the fluid controller 12, or at least use the switch to manually override its operation.

In a further embodiment, the switch 38 may be used in conjunction with the fluid controller. In other words, it may include an actual means 40 for actuation of the switch 38 between the “ON” and “OFF” positions. The actuation means 40 can include a sensor 42 positioned proximal to the first end 18 of the first catheter 14. The sensor 42 can be affixed to an outer or inner surface of the first catheter 14, or in the alternative, positioned in the cranial space 16 offset from the first catheter 14. The sensor 42 is operably connected to the switch 38 via a wire 44.

The sensor 42 can be, for example, a pressure sensor and used to determine when and how long the device should operate in a first cycle, a second cycle, both cycles, or in a dormant state. The pressure sensor 42 may operate to measure the pressure in the cranial space 16, and may be configured such that when the pressure in the cranial space exceeds a first “threshold” pressure a signal is sent to the switch 38, actuating the switch 38 from a first position, “OFF” position, to a second position, “ON” position, turning on the pump 30 to remove fluid from the cranial space 16. When the fluid pressure in the cranial space 16 is decreased to become less than a second “threshold” pressure, the sensor 42 signal is discontinued, such that, the switch 38 actuates from the second position, “ON” position, to the first position, “OFF” position, shutting off the pump 30 and discontinuing the removal of the fluid from the cranial space 16. The first and second threshold pressures may be substantially similar pressure levels, or alternatively may differ in amount where the second threshold pressure is less than the first. In this manner, activation of the first and second cycles may begin once a certain pressure threshold is reached and may continue until the pressure levels return to below a desired amount.

Thus, when the fluid pressure in the cranial space 16 is decreased to become less than the “threshold” pressure, the sensor 42 sends a second signal to the switch 38, actuating the switch 38 from the second position, “ON” position, to the first position, “OFF” position, shutting off the pump 30 and discontinuing the removal of the fluid from the cranial space 16.

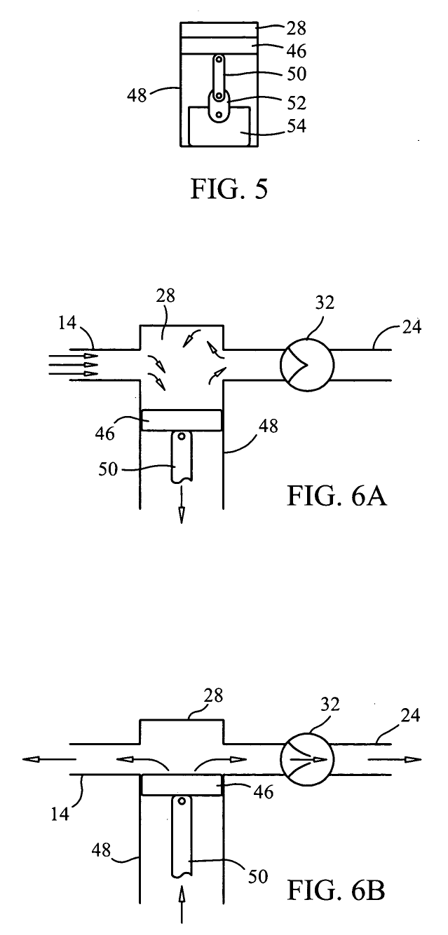

Referring to FIG. 5, an exemplary pump 30 includes a piston 46 slidably positioned in a cylinder 48, a portion of which includes the fluid reservoir 28. The piston 48 is pivotally connected to an end of a shaft 50. The opposite end of the shaft 50 is pivotally connected to a cam 52, where the cam 52 is connected to a motor 54. The motor 54 acts to rotate the cam 52, such that the shaft 50 and piston 46 move through an “up” stroke and a “down” stroke within the cylinder 48.

Referring to FIG. 6A, on the “down” stoke of the shaft 50 and piston 46 are drawn out of the fluid reservoir 28, drawing a vacuum in the fluid reservoir 28, such that the pressure in the fluid reservoir 28 is less than the pressure in the cranial space 16. As a result, fluid is drawn into the fluid reservoir 28 from the cranial space 16 through the first catheter 14. In a likewise manner, the pressure in the fluid reservoir 28 is less than the pressure in the second catheter 24. As a result, the one-way vale 32 is maintained in a closed position, preventing fluid from entering or exiting the fluid reservoir 28 through the second catheter 24.

Referring to FIG. 6B, on the “up” stroke the shaft 50 and piston 46 are moved into the fluid reservoir 28, pressurizing the fluid reservoir 28, such that the pressure in the fluid reservoir 28 is greater than the pressure in the cranial space 16 and the second catheter 24. The pressure in the fluid reservoir 28 is sufficiently high to open the one-way valve 32. In this manner the fluid is forced out of the fluid reservoir 28, where a portion of the fluid is forced out through the second valve 32 and the second catheter 24. Additionally, a portion of the fluid is forced out of the fluid reservoir 28 through the first catheter 14, back into the cranial space 16. In this manner, the first catheter 14 is continually back flushed, which can substantially prevent the accumulation of particulate matter the first catheter 14. When activated, the pump 28 is continually operated between the “up” and “down” strokes, to remove fluid from the cranial space 16.

The above described bladder and pump are only exemplary devices and it is contemplated that other bladder and pump devices know in the art that are operable between first and second cycles to draw in and expel fluid can be utilized in the present invention.

All references cited herein are expressly incorporated by reference in their entirety.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An implantable device for removing fluid from a first location in a body of a patient comprising:
a first catheter including a first end positionable within the first location in the body of the patient and a second end;
a second catheter including a first end and a second end;
a reservoir in fluid communication with the second end of the first catheter and the first end of the second catheter;
a one way valve interposed between the reservoir and the first end of the second catheter; and
a pump connected to the reservoir and operable between a first and second cycle, wherein in the first cycle the fluid is drawn in through the first catheter into the reservoir and in the second cycle a first portion of the fluid is forced out of the reservoir through the first catheter into the first location and a second portion of the fluid is forced out of the reservoir through the one-way valve and the second catheter.

2. An implantable device as set forth in claim 1 further comprising a power source connected to the pump.

3. An implantable device as set forth in claim 2 wherein the power source is a battery.

4. An implantable device as set forth in claim 2 further comprising a switch interposed between the pump and the power source.

5. An implantable device as set forth in claim 4 wherein the switch comprises a manual override for operation of the pump.

6. An implantable device as set forth in claim 4 further comprising an actuation means connected to the switch.

7. An implantable device as set forth in claim 6 wherein the actuation means includes a sensor.

8. An implantable device as set forth in claim 1 further comprising a filter interposed between the reservoir and the first catheter.

9. An implantable device as set forth in claim 1 wherein the one way valve prevent fluid from entering or exiting the reservoir in the first cycle.

10. An implantable device as set forth in claim 1 wherein the second end of the second catheter is positioned in a second body portion.

11. A subcutaneously implantable device for removing fluid from a first location in a body of a patient comprising a fluid controller including a fluid inlet and a fluid outlet and having first and second operation cycles, wherein during the first operation cycle the fluid controller draws fluid in through the fluid inlet and during the second cycle the fluid controller expels a first portion of the fluid out through the fluid inlet and a second portion of the fluid out through the fluid outlet.

12. A subcutaneously implantable device as set forth in claim 11 further comprising a one-way valve connected to the fluid outlet, wherein the one way valve prevents fluid from entering or exiting the fluid controller through the fluid outlet in the first cycle.

13. A subcutaneously implantable device as set forth in claim 12 wherein the fluid controller further comprises:
a reservoir in fluid communication with the fluid inlet and the fluid outlet; and
a pump connected to the reservoir and operable between the first and second cycles.

14. A subcutaneously implantable device as set forth in claim 13 further comprising:
a power source; and
an switching means connecting the power source and the pump.

15. A subcutaneously implantable device as set forth in claim 14 wherein the switch means includes a manual override for operation of the pump.

16. A subcutaneously implantable device as set forth in claim 14 wherein the switching means comprises;
a sensor; and
a switch operably connected to the sensor.

17. A method of draining a fluid from a first location in a body of a patient comprising:
positioning a shunt system in fluid communication with the first location in the body of the patient;
draining the fluid from the first location in the body of the patient into the shunt system; and
expelling the fluid from the shunting system, wherein a first portion of the fluid is expelled from the shunt system into the first location in the body of the patient and a second portion of the fluid is expelled from the shunt system to a second location different from the first location.

18. A method as set forth in claim 17, wherein draining the fluid from the first location in the body of the patient into the shunt system comprises operating the shunt system to drawn fluid into the shunt system through a first catheter positioned in the first location in the body of the patient.

19. A method as set forth in claim 18, wherein expelling the fluid from the shunting system comprises operating the shunt system to expel the first portion of the fluid through the first catheter into the first location in the body of the patient and to expel the second portion of the fluid through a second catheter into the second location.

20. A method as set forth in claim 19, further comprising preventing fluid from entering or exiting the shunt system through the second catheter when the shunt system is operated to draw the fluid from the first location in the body of the patient.
